(19) United States
(12) Patent Application Publication
(10) Pub. No.: US 2008/0045842 A1
(43) Pub. Date: Feb. 21, 2008

(54) Conformable tissue contact catheter

(75) Inventor: Simon M. Furnish, New York, NY (US)

Correspondence Address: PATTON BOGGS LLP, 8484 WESTPARK DRIVE, SUITE 900, MCLEAN, VA, 22102, US

(73) Assignee: Prescient Medical, Inc., Doylestown, PA (US)

(21) Appl. No.: 11/878,033

(22) Filed: Jul. 20, 2007

Related U.S. Application Data
(60) Provisional application No. 60/832,158, filed on Jul. 21, 2006.

(51) Int. Cl. A61B 6/12

(52) U.S. Cl. 600/478

(57) ABSTRACT

The invention provides basket-style catheter probes that are configured to automatically radially expand or contract in response to widening or narrowing of a lumen so that contact with the lumen wall is maintained during probing. Related diagnostic systems and methods are also provided.

### CROSS-REFERENCE TO RELATED APPLICATIONS

This application claims the benefit of U.S. provisional application Ser. No. 60/832,158 filed Jul. 21, 2006, which is hereby incorporated by reference in its entirety.

### FIELD OF THE INVENTION

The invention relates to the field of side-viewing catheter probes and more specifically to basket-style catheter probes.

### BACKGROUND OF INVENTION

Existing “basket-style” multi-arm tissue contact catheters have been described in prior disclosures by the inventor and others, such as U.S. Pub. No. 2005/0107706, which is incorporated by reference herein in its entirety. These include various multi-arm tissue contact catheters, including various possible embodiments for deployment and retraction of the basket arms for easier delivery and compatibility with differently sized vessels.

The particular configuration of the scanning core of a basket-style catheter may include single or multiple optical fibers for the transmission and/or collection of light from the side walls of a vessel. Alternatively, the scanning core may be an ultrasonic transducer or transducer array. A third option is the combination of optics and ultrasound, combining the best features from gross morpohologic measurements (as with IVUS), fine morphologic measurements (as with OCT and variants) as well as analysis of chemical composition using one of the many available modes of tissue spectroscopy (as with Raman spectroscopy, diffuse reflectance, etc.). Other options include, but are not limited to, small temperature transducers (e.g., a thermocouple or RTD thermometer probe) for measuring tissue temperature at the site of contact (thermography). Various basket and umbrella-style tissue contact catheters have been designed and manufactured for intravascular thermography and already exist in the prior art. Intravascular magnetic resonance imaging (MRI) is another possible detection modality that could be well suited for tissue-contact style catheters.

U.S. Pat. No. 6,522,913 discloses systems and methods for visualizing tissue during diagnostic or therapeutic procedures that utilize a support structure that brings sensors into contact with the lumen wall of a blood vessel, and is incorporated by reference herein in its entirety

U.S. Pat. No. 6,701,181 discloses multi-path optical catheters, and is incorporated by reference herein in its entirety.

U.S. Pat. No. 6,873,868 discloses multi-fiber catheter probe arrangements for tissue analysis or treatment, and is incorporated by reference herein in its entirety.

U.S. Pat. No. 6,949,072 discloses devices for vulnerable plaque detection, and is incorporated by reference herein in its entirety.

U.S. Publication No. 2002/0183622 discloses a fiber-optic apparatus and method for the optical imaging of tissue samples, and is incorporated by reference herein in its entirety.

U.S. Publication No. 2003/0125630 discloses catheter probe arrangements for tissue analysis by radiant energy delivery and radiant energy collection, and is incorporated by reference herein in its entirety.

U.S. Publication No. 2004/00176699 discloses basket-type thermography catheters in which each probe arm is independently moveable, and is incorporated by reference herein in its entirety.

U.S. Publication No. 2004/0204651 discloses infrared endoscopic balloon probes, and is incorporated by reference herein in its entirety.

U.S. Publication No. 2004/0260182 discloses intraluminal spectroscope devices with wall-contacting probes, and is incorporated by reference herein in its entirety.

U.S. Publication No. 2005/0054934 discloses an optical catheter with dual-stage beam redirector, and is incorporated by reference herein in its entirety.

U.S. Publication No. 2005/0075574 discloses devices for vulnerable plaque detection that utilize optical fiber temperature sensors, and is incorporated by reference herein in its entirety.

U.S. Publication No. 2005/0165315 discloses a side-firing fiber-optic array probe, and is incorporated by reference herein in its entirety.

U.S. Publication No. 2006/0139633 discloses the use of high wavenumber Raman spectroscopy for evaluating tissue, and is incorporated by reference herein in its entirety.

The basket-style catheters known in the art have limitations due to their complexity of construction, limited flexibility and lack of atraumatic conformability with tortuous vessels. While deployable baskets appear to address some compatibility issues, they are quite complex, with many moving parts and sliding required over a long distance within a very small area and could subject vessels to unsafe radial forces when compressed significantly. What is needed is a new type of tissue contact probe that is compact and flexible, yet unhindered by elaborate design elements.

### SUMMARY OF INVENTION

One embodiment of the invention provides a flexible intravascular catheter for performing analysis of a blood vessel wall that includes: an elongate catheter body having a proximal end and a distal insertion end; a guidewire lumen, and an interrogation section disposed near the distal insertion end, wherein the interrogation section comprises at least two probe arms, each probe arm including an optical probe apparatus or other type of probe apparatus or sensor disposed in a flexible tube that is radially bowed or bowable outward from the central axis of the catheter to contact or near a blood vessel wall. The distal-most portion of the arms are gathered together, clustered around and connected to a sliding element, such as ring or tube segment, which freely slides back and forth along the guidewire, thereby allowing the basket to flexibly contract in response to compression in smaller vessels and flexibly expand when encountering widening in vessels. This distal portion is only connected to the proximal catheter segment via the flexible probe arms and can be tailored for achieving extremely low radial forces under full compression of the probe arms.

A further embodiment of the invention includes a pre-shaped spring support structure for the probe arms, e.g., extending through, with or forming the probe arms, which biases the basket to a particular maximum diameter and preferred profile. The pre-shaped spring support may, for example, be fabricated from metallic and/or polymeric materials. The support spring may, for example, be fabricated from a stainless steel, spring steel or Nitinol round or flat wire. Optionally, a molded or laser cut component could be fabricated to create a monolithic body consisting of leaf springs and the distal guidewire lumen. A further option utilizes laser cut, heat treated and electropolished tubing or wire form assembly fabricated into a flexible self-expanding stent-like structure. Still another option is to utilize one or more plastic (polymer) materials, such as liquid-crystal polymers, acrylic, acrylonitrile-butadiene styrene (ABS), polycarbonate (PC), poly-ether ether-ketone (PEEK) or other resins. A polymeric support structure may be formed by any method, such as molding, heat forming, extrusion, dip-coating and/or machining to yield a suitable geometry. Each arm may be thin and contoured to provide the desired radial force and may also be utilized as a structural element for attachment of the scanning element. A support/reinforcement member may be provided, as shown in FIGS. 5A-C and further described below. A tapered profile to each arm may also be beneficial to provide favorable compression behavior.

Another embodiment of the invention provides a conformable multi arm catheter, that includes: a proximal end; a distal end; a central axis; a proximal catheter segment with a guidewire lumen for a guidewire; a distal interrogation section extending from the distal end of the proximal catheter segment, wherein the interrogation section includes at least two flexible probe arms, that in an unconstrained state, radially bow out from the central axis and then, proceeding distally, bow back toward the central axis of the catheter; and a distal insertion segment connected to the distal ends of the probe arms and providing a guidewire lumen so that the distal insertion segment is slideably engageable with the guidewire. At least one and preferably at least two of the probe arms may include a probe element, for example, a scanning core, disposed at a position along the probe arm to interrogate a target when the probe arms are radially extended to contact or near an interrogation target.

Still another embodiment of the invention provides an improved basket-style catheter having a catheter shaft, and a distal basket section having a proximal end segment, a distal end segment and at least two probe arms connecting the proximal and distal ends, in which the improvement includes the distal end segment being configured for slideable engagement with the guidewire to permit radial expansion and contraction of the probe arms in response to changes in the diameter of a lumen in which the basket section is disposed.

A related embodiment of the invention provides an improved basket-style catheter assemblage that includes a (i.) catheter shaft, (ii.) a distal basket section having a proximal end segment, a distal end segment and at least two probe arms connecting the proximal and distal ends, and (iii.) a guidewire, in which the improvement includes the distal end segment being configured for slideable engagement with the guidewire to permit radial expansion and contraction of the probe arms in response to changes in the diameter of a lumen in which the basket section is disposed.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIG. 1 shows a 4-channel, basket-type catheter with a floating distal segment.

FIG. 2 shows the distal end basket detail of the catheter design.

FIG. 3 shows a side view of the distal end basket detail at two positions within a tapering vessel. The basket is shown conforming to varying vessel geometry.

FIG. 4 shows a front view of the unconstrained and compressed basket.

FIG. 5 shows a basket structural reinforcement element.

FIG. 6 shows a catheter embodiment of the invention that includes a longitudinally displaceable control sheath for controlling the radial extension of the probe arms of the catheter.

FIG. 7 shows Raman spectra of cholesterol and various cholesterol esters in the Raman high wavenumber region.

### DETAILED DESCRIPTION

The invention provides basket-style catheter probes that are configured to automatically radially expand or contract in response to widening or narrowing of a body lumen so that contact with the lumen wall is maintained as the probe is traverses the lumen. The basket segment of the probe has a lumen that accommodates a guidewire through its length and includes a proximal end that remains static with respect to the catheter to which it is attached and a distal end that slideably surrounds the guidewire. Positioned between, and attached, to the each of the proximal and distal ends are probe arms that have an outward radial bias so that their tendency is to flex toward a lumen wall. The slideable distal end of the basket segment permits radial expansion and contraction of the probe arms by way of changes in the distance between the proximal and distal ends of the probe as the probe travels within a lumen.

The basket-style probe assemblies of the invention provide for the delivery and/or collection of diagnostic and/or therapeutic energy in small spaces. The probe assemblies can be small and flexible and are well-suited to performing minimally invasive diagnostic examinations of biological tissues in vivo.

The invention is further described below with reference to the appended figures.

As referred to herein, the term “probe arm” means one of the flexible elements that is disposed between the proximal end and distal end of the basket section and which contacts or nears a lumen wall, such as an artery wall, by radial expansion. One or more of the probe arms may include an operable probe element or sensor, also referred to as a scanning core herein, for delivering and/or receiving diagnostic or therapeutic energy, for example, light, ultrasound or heat. A 4-channel basket catheter profile is shown in the figures. However, catheters of the invention may generally have at least two probe arms and may, for example, have 2, 3, 4, 5, 6, 7 or 8 probe arms. By using multiple radially spaced probe arms, a composite radial field-of-view can be built up. The probe arms may be at least substantially uniformly radially spaced. The 6-probe arm configuration provides an excellent balance of radial coverage for optical interrogation and maneuverability, in a catheter sized for interrogation of human, e.g., adult, coronary arteries.

The particular configuration shown in the accompanying figures is an “over the wire” catheter with a guidewire lumen passing the entire length of the catheter, and out through the “guidewire port” on the hub. For simplicity, the remaining descriptions will discuss the embodiment as an optical spectroscopy catheter, but the invention is not limited to this modality and may, for example, be additionally or alternatively implemented with other diagnostic modalities such as ultrasound (IVUS), MRI, OCT or thermography.

The optical fiber bundles may begin within each distal scanning optic core and extend to proximal to connectors which interface with a light source and detector. Each optical fiber bundle may contain one or more optical fibers.

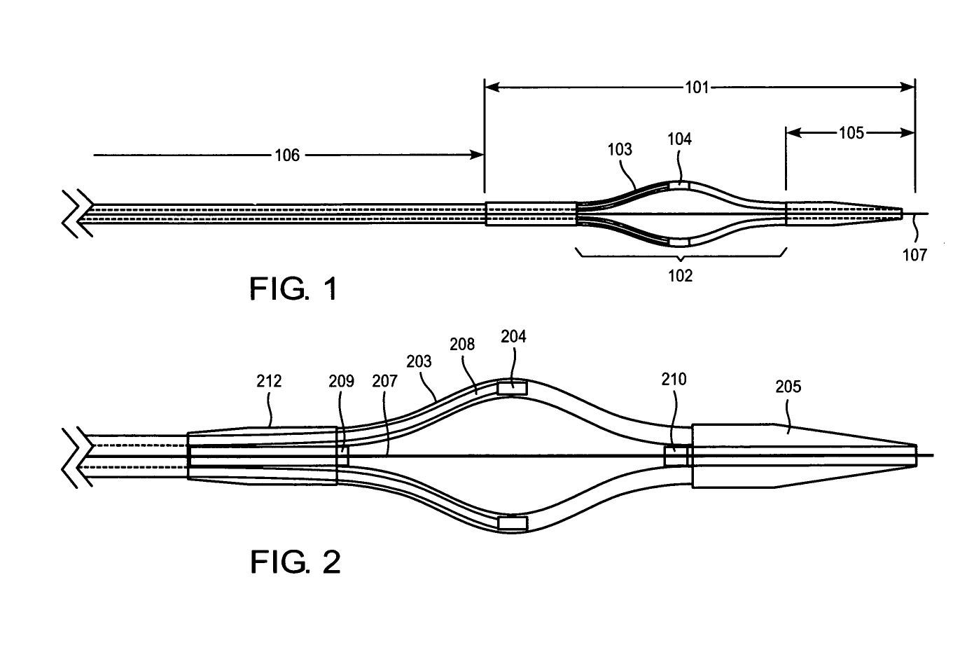

FIG. 1 shows the various segments of a catheter embodiment of the invention. Distal segment 101 of the catheter includes basket section 102 that includes probe arms 103 (two are shown), which include a distal scanning core 104 having one or more side-viewing probes. The distal ends of the probe arms are connected to distal tip segment 105. Guidewire 107 is seen extending through the length of the catheter, through the basket section, and into and out of distal tip segment 107. At the proximal end of distal segment 101 is an optional retaining sleeve 112 surrounding the basket probe arms. Proximal to distal segment 101 is the proximal shaft segment 106 of the catheter.

FIG. 2 shows further details with respect to the indwelling end of the catheter shown in FIG. 1. The side arms in this view have been partially removed to reveal the guidewire and sliding distal segment. Each probe arm 203 has a scanning core 204 that is operably connected to at least one optical fiber or lead wire 208 to permit light/signals to be delivered to a target and transmitted out of the catheter for analysis. Collected light/signals may optionally be multiplexed by a multiplexer at some point before running the entire length of the catheter. In the embodiment shown, proximal guidewire lumen tubing 209 can be seen enclosed by retaining sleeve 212. At the proximal end of distal tip 205, distal guidewire lumen tubing 201 can be seen.

U.S. Publication No. 2004/00176699 teaches embodiments of a basket type thermography catheter in which the distal ends of each probe arm are independently slideably engaged with the distal tip segment of the catheter. In contrast, the catheters of the present invention are preferably configured to perform optical spectroscopy, such as Raman spectroscopy, such as high wavenumber Raman spectroscopy. Thus, according to the present invention, each probe arm may contain at least one optical fiber for side/lateral-viewing from the scanning core regions of the probe arms. Further in contrast to U.S. Publication No. 2004/00176699, according to the present invention, the distal end of each probe arm may be fixably connected (have a fixed connection point) to or integrated with the distal tip segment of the catheter, thereby simplifying construction and operation of the catheter.

For optical probe elements, a lateral field-of-view may be provided by any suitable means, for example, by using a mirror or prism in optical communication with the one or more optical fibers and/or by using angle-cut optical fiber faces. For example, a 45-degree mirror or prism may be used to laterally redirect light with respect to a distal scanning core of a probe.

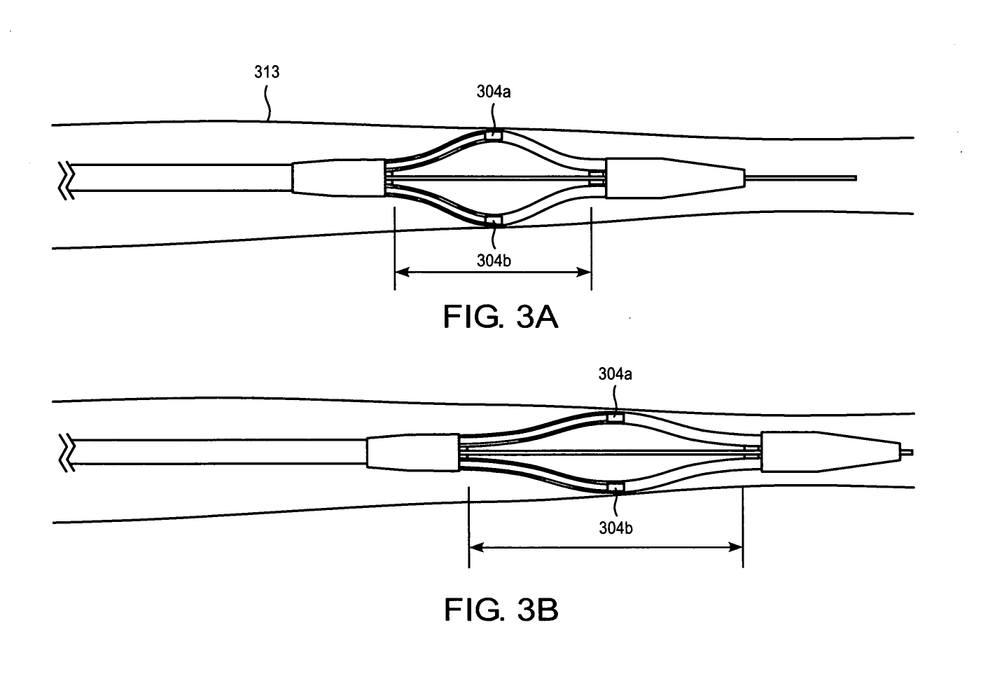

FIG. 3 shows the indwelling end of the catheter positioned within a narrowing vessel 313 in two positions as, for example, during a pullback sequence. As the catheter encounters a larger vessel diameter, as shown in FIG. 3A, the basket expands to conform to the vessel to maintain scanner contact with the vessel walls. As the basket encounters a narrower vessel diameter, as in FIG. 3B, the basket radially retracts. As the basket expands and contracts, the distal segment slides back and forth along the guidewire to accommodate the change in basket length. Thus, the scanning cores 304a and 304b remain close to the lumen wall for interrogation as the distal end of the catheter traverses the lumen. The “floating” distal end segment of the catheter may be distally tapered as shown or may have other configurations.

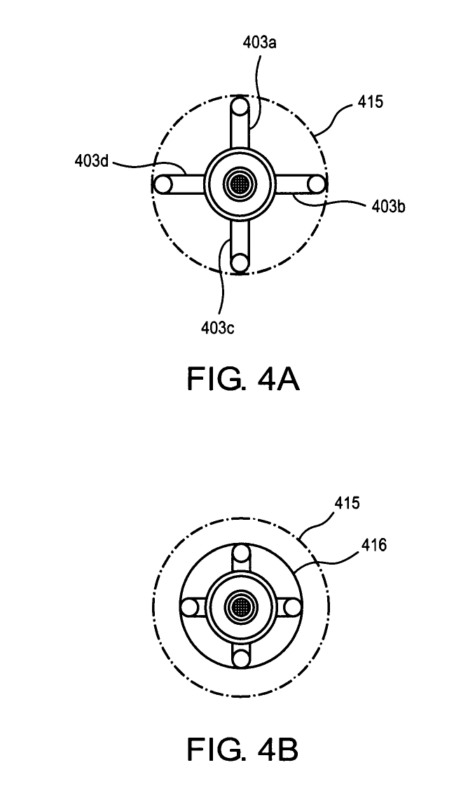

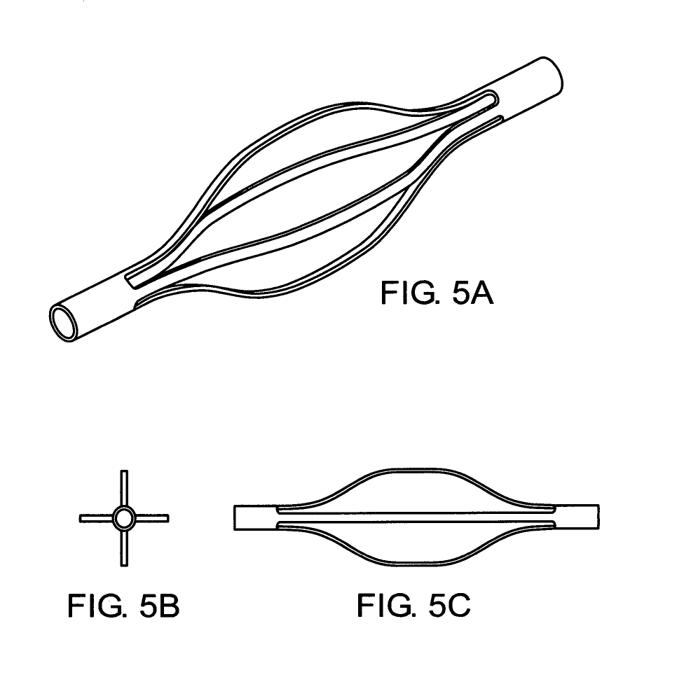

FIGS. 4A and 4B show a front (end-on) view of the unconstrained basket and a compressed basket with a reduced total profile, respectively. Four probe arms 403a-d are shown. In FIG. 4A probe arms 403a-d are unconstrained and maximally radially extended to a radius shown by bounding circle 415. FIG. 4B shows the basket arms constrained by the smaller radius of a vessel, indicated by circle 416.

The outward radial shape or “bias” of the probe arms for tissue contact may, for example, be obtained by utilizing probe arms with a pre-set curvature. For example, the probe arms may be formed from plastic tubing or segments having a curvature that provides the outward radial shape for tissue contact. Another approach is to provide this support via an internal structural element. FIG. 5 shows a basket reinforcement element formed from a slotted tube used to reinforce the desired shape of the catheter basket in the unconstrained shape. The support/reinforcement member may be a unitary structure, i.e., a one-piece structure, as shown. The embodiment of FIG. 5 is a unitary support/reinforcement structure having four arms with straight, flat arm profiles. FIG. 5A shows an isometric view of the support structure. FIG. 5B shows an end-on view of the support structure. FIG. 5C shows a side-view of the support structure. The tube may, for example, be made from a stainless steel, a spring steel, superelastic Nitinol alloy or a polymeric material such as PEEK, Polyimide, Polyamide, PTFE or other engineering materials for medical device construction. The basket reinforcement element tube may, for example be fabricated in a collapsed form (laser cut thin-walled tubing) and then compressed (with respect to its lateral axis) within a mold base and heat treated to set the preferred unconstrained shape. Injection molding or thermoforming of plastic/polymer materials may also be used to fabricate the basket reinforcement element.

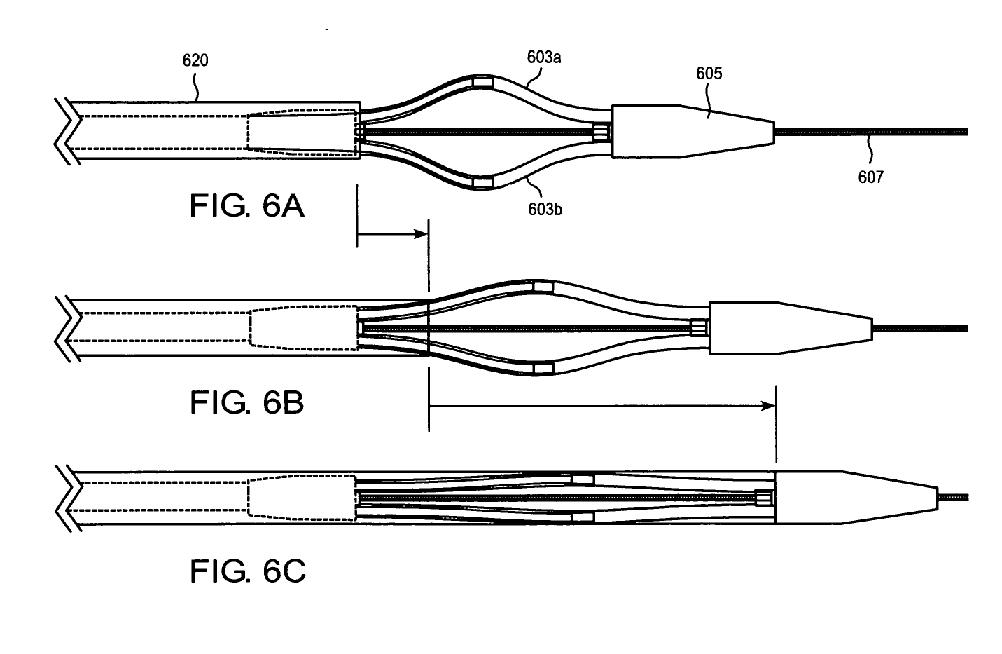

FIG. 6 shows an embodiment of the invention that is similar to the catheters shown in FIGS. 1-4, but which further includes a longitudinally displaceable control sheath 620 for controlling the radial extension of the probe arms of the catheter. Two probe arms 603a and 603b are indicated in the figure and guidewire 607 can be seen extending through the basket section and out the distal tip segment 605 of the catheter. FIG. 6A shows control sheath 620 withdrawn to a position where its distal end is disposed around the proximal end of the basket section. In this position the, the probe arms of the basket section are free to radially extend toward their maximum radius and adjust to a radius determined by the dimension of a lumen in which the basket section is disposed. FIG. 6B shows control sheath 620 advanced distally versus FIG. 6A. In this position, the diameter of control sheath 620 partially restrains the radial extension of the probe arms of the basket section. FIG. 6C shows control sheath 620 advanced further distally so that its distal end meets the proximal end of distal tip segment 605. In this position, the radial extension of the probe arms is completely restrained and the probe arms are completely enclosed within sheath 620. Thus, the deployment and radial extension of the probe arms of the basket section may be controlled by advancing and withdrawing control sheath 620. The lateral displacement of control sheath 620 may be controlled or actuated from the proximal end of the catheter outside a patient's body.

The invention also provides a method for diagnostically interrogating and/or treating a body lumen wall, such as blood vessel lumen wall, that includes the steps: of inserting a catheter according to the invention into a body lumen, such as a blood vessel lumen; and delivering diagnostic and/or therapeutic energy via at least one probe element on at least one probe arm of the catheter to the lumen wall. The energy may for example, be light energy. Energy received via the probe elements or measured by the probe elements may be analyzed to evaluate and diagnose a subject tissue. The invention is not limited by the method used to interrogate and diagnosis the condition of a blood vessel wall. Optical and/or non-optical methods may be used. Multiple methods may also be used. Suitable optical methods include, but are not limited to, low-resolution and high resolution Raman spectroscopy, fluorescence spectroscopy, such as time-resolved laser-induced fluorescence spectroscopy, and laser speckle spectroscopy. Photoacoustic stimulation in conjunction with acoustical detection by any means may also be used. One embodiment of the invention is a method for diagnosing and/or locating one or more atherosclerotic lesions, such as vulnerable plaque lesions, in a blood vessel, such as a coronary artery of a subject, using a catheter as described herein to evaluate the properties of a vessel wall, such an artery, at one more locations along the vessel. In any of the embodiments, the catheter including its basket section and probe arms thereof may be sized for interrogation of human coronary arteries.

Differentially diagnosing, identifying and/or determining the location of an atherosclerotic plaque, such as a vulnerable plaque, in a blood vessel of a patient can be performed by any method or combination of methods. For example, catheter-based systems and methods for diagnosing and locating vulnerable plaques can be used, such as those employing optical coherent tomography (“OCT”) imaging, temperature sensing for temperature differentials characteristic of vulnerable plaque versus healthy vasculature, labeling/marking vulnerable plaques with a marker substance that preferentially labels such plaques, infrared elastic scattering spectroscopy, and infrared Raman spectroscopy (IR inelastic scattering spectroscopy). U.S. Publication No. 2004/0267110 discloses a suitable OCT system and is hereby incorporated by reference herein in its entirety. Raman spectroscopy-based methods and systems are disclosed, for example, in: U.S. Pat. Nos. 5,293,872; 6,208,887; and 6,690,966; and in U.S. Publication No. 2004/0073120, each of which is hereby incorporated by reference herein in its entirety. Infrared elastic scattering based methods and systems for detecting vulnerable plaques are disclosed, for example, in U.S. Pat. No. 6,816,743 and U.S. Publication No. 2004/0111016, each of which is hereby incorporated by reference herein in its entirety. Time-resolved laser-induced fluorescence methods for characterizing atherosclerotic lesions are disclosed in U.S. Pat. No. 6,272,376, which is incorporated by reference herein in its entirety. Temperature sensing based methods and systems for detecting vulnerable plaques are disclosed, for example, in: U.S. Pat. Nos. 6,450,971; 6,514,214; 6,575,623; 6,673,066; and 6,694,181; and in U.S. Publication No. 2002/0071474, each of which is hereby incorporated by reference herein in its entirety. A method and system for detecting and localizing vulnerable plaques based on the detection of biomarkers is disclosed in U.S. Pat. No. 6,860,851, which is hereby incorporated by reference herein in its entirety.

Raman spectroscopy has proven capable of determining the chemical composition of tissues and diagnosing human atherosclerotic plaques. Typical methods of collecting Raman scattered light from the surfaces of artery do not register information about how far the scattering element is from the collection optics. Two wavenumber regions that yield useful information for evaluating the condition of blood vessels are the so-called Raman fingerprint region i.e., approximately 200 to 2,000 cm⁻¹, and the so-called high wavenumber region, i.e., approximately 2,600 to 3,200 cm⁻¹. The collection of Raman spectra in the fingerprint (FP) region, through optical fibers is complicated by Raman “background” signal from the fibers themselves. In order to collect uncorrupted FP spectra, complicated optics and filters on the tips of catheters and often these designs require the use of multiple fibers. Since the Raman scattered signal is weak, large multimode fibers are utilized in the multi-fiber catheter designs, which creates an unwieldy catheter that is less than optimal for exploring delicate arteries, such as the human coronary arteries. However, common optical fiber materials generate very little Raman background signal in the high wavenumber region, permitting a simplified, single optical fiber probe element implementation of intravascular Raman spectroscopy.

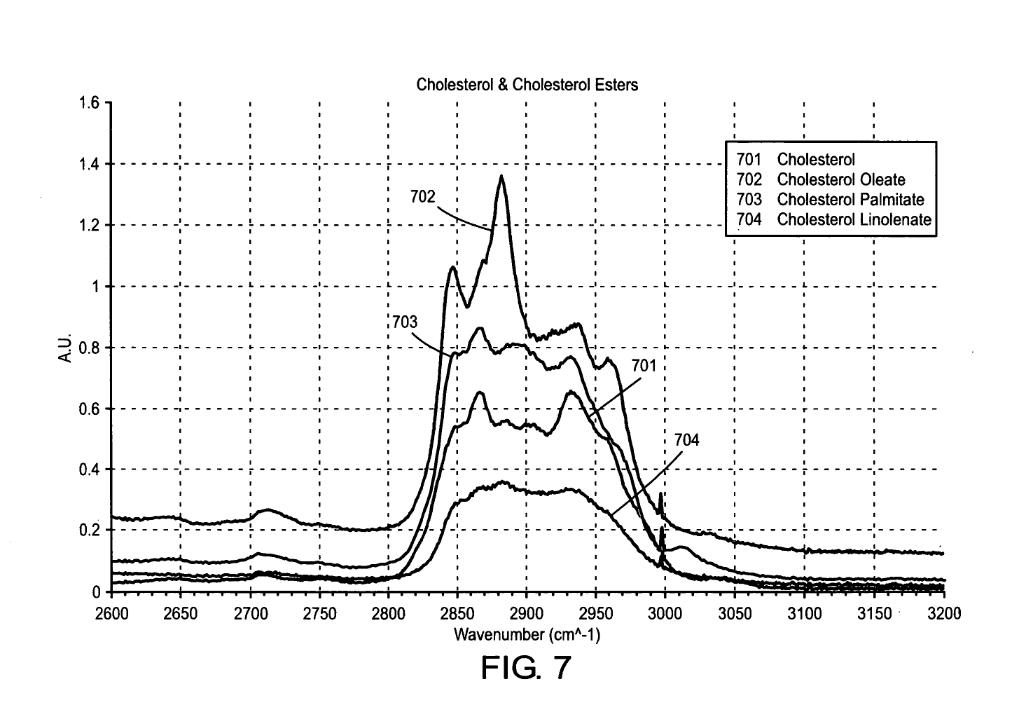

Since cholesterol and its esters have distinctive Raman scattering profiles within the Raman high wavenumber region, the use of the Raman high wavenumber region for analysis is particularly useful for locating and characterizing lipid-rich deposits or lesions as may occur in blood vessels, such a vulnerable plaques in arteries, such as the coronary arteries. FIG. 7 shows Raman spectra of cholesterol and cholesterol esters in the high wavenumber region. Specifically, curve 701 is a Raman spectrum for cholesterol, curve 702 is a Raman spectrum for cholesterol oleate, curve 703 is a Raman spectrum for cholesterol palmitate and curve 704 is a Raman spectrum for cholesterol linolenate.

One embodiment of the invention provides a method for evaluating the wall of a blood vessel such an artery, such as a coronary artery, such as a human coronary artery, that includes the steps of:

providing any of the intravascular basket catheter embodiments of the invention having a proximal end and a distal insertion end including a basket section comprising at least two radially extendable wall-contacting optical probe arms;

disposing the basket section of the catheter in a blood vessel; and

taking optical readings of the vessel wall at one or more locations in the blood vessel using the at least two optical probe arms.

In one variation, the method includes transmitting light, such as laser light, from a light source to target regions of a lumen wall, such as a blood vessel wall, via the scanning core of the probe arms of the catheter and collecting and analyzing inelastically scattered (Raman scattered) light resulting from the illumination of the target regions using a Raman spectrometer. The Raman spectrometer may be configured to measure Raman scattered light in the high wavenumber region and/or the fingerprint region and the data for either or both of the regions may be analyzed to determine the chemical composition of the target regions and/or diagnose the target regions/tissue.

In another variation, the method includes transmitting light, such as laser light, for fluorescence stimulation of the target regions of a lumen wall, such as a blood vessel wall, via the scanning core of the probe arms of the catheter and collecting and analyzing fluorescent emissions resulting from the illumination of the target regions using a fluorescence spectrometer. In a sub-variation, time-resolved laser-induce fluorescence is performed using a catheter embodiment of the invention.

It should be understood for the above methods that the probe arms are radially extended to contact or closely near the vessel walls in order to take readings from the probe arm-disposed optical assemblies. The step of taking readings may include taking the recited readings at more than one longitudinal location in a blood vessel, for example, while the catheter is pulled back by operation of a catheter pullback mechanism.

The invention also provides an integrated system for evaluating the status of a lumen wall such as a blood vessel wall, for example, for diagnosing and/or locating vulnerable plaque lesions, that includes a basket-style catheter according to the invention having probe elements (scanning cores) for interrogating the lumen wall that are in communication with an analyzer for analyzing the signal and/or information received via the probe elements. The analyzer may include a computer.

A related embodiment provides an integrated system for optically evaluating the status of a lumen wall, such as a blood vessel wall, for example, for diagnosing and/or locating atherosclerotic lesions, such as vulnerable plaque lesions in an artery, that includes an basket-style catheter according to the invention having optical probe elements for interrogating the blood vessel in communication with a light source such as a laser for illuminating a target region of a blood vessel via the catheter and a light analyzer, such as a spectrometer, for analyzing the properties of light received from the target region via the catheter.

A related embodiment of the invention provides a diagnostic catheter system for the evaluation of blood vessel walls that includes an intravascular diagnostic catheter as described herein, a light source such as a laser for stimulating Raman scattered light emissions from a target region via the wall-contacting portion (scanning core) of the probe arms of the catheter, and a Raman spectrometer for analyzing Raman scattered light collected from a target via the wall-contacting portion of the probe arms of the catheter. The system may be configured to collect and analyze Raman spectral data within the region of approximately 2,600 to 3,200 cm⁻¹, i.e., the so-called high wavenumber region, and/or the within the region of approximately 200 to 2,000 cm⁻¹, i.e., the so-called fingerprint region. The optical probe arms may, for example, each have a single optical fiber and the system may be configured to perform high wavenumber Raman spectroscopy from each probe arm via the single optical fiber.

One or more computers, or computer processors generally working in conjunction with computer accessible memory, may be part of any of the systems for controlling the components of the system and/or for analyzing information obtained by the system.

Co-owned U.S. provisional application Ser. No. 60/853,427 which is hereby incorporated by reference in its entirety, teaches windowless optical probe assemblies for use with Raman spectroscopy, such as high wavenumber Raman spectroscopy, which may be implemented with the present invention. In accordance with this application, the probe arms may include one or more optical fibers housed in material(s) having a very low Raman scattering cross-section in the wavenumber region used for analysis of a target and being adequately transparent to excitation light delivered via the fiber optics to the target and to Raman-scattered light (inelastically scattered light) collected from the irradiated target in the desired wavenumber range. In this manner, a separate window or aperture is not needed in the viewing portion (scanning core region) of the probe arm to permit illumination and collection of light having the desired ranges of wavelengths, thereby simplifying manufacture and improving performance of the catheter.

Thus, in any of the embodiment of the present invention, at least one of the probe arms may include: an optical fiber assembly having a viewing portion (scanning core portion) for transmitting and receiving light, wherein at least the viewing portion of the optical fiber assembly is enclosed in a material, such as a polymeric material, having an at least substantially non-discernable Raman scattering signal (a level not interfering with analysis) in one or more preselected wavenumber regions used for analysis of a target and being adequately transparent to excitation light delivered via the optical fiber assembly to the target and to Raman-scattered light collected from the irradiated target in the preselected wavenumber range by the optical fiber assembly. The optical fiber assemblies of the probes and catheter probes may include one or more optical fibers. In one variation, the main bodies of the probe arms (excluding the optical fiber assemblies) may be entirely composed of or enclosed in the polymeric material. The preselected wavenumber region may, for example, be in the range of approximately 2,600 to 3,200 cm−1, i.e., within the high wavenumber region. For the high wavenumber region, the enclosure material may, for example, include or consist of polymer material that at least substantially does not include carbon-hydrogen bonds, such as polytetrafluoroethylene (PTFE), fluorinated ethylene-propylene (FEP) and perfluoroalkoxy polymer resin (PFA). In these cases, the excitation wavelength used to obtain the high wavenumber spectra may, for example, be at or around 740 nm, or at a suitable near infrared wavelength generally. The light source may be a laser, such as a wavelength stabilized multi-mode laser diode, such as a Volume Bragg Grating Stabilized multi-mode laser diode (available, e.g., from PD-LD, Inc., Pennington, N.J.)

Each of the patents and other publications cited in this disclosure is incorporated by reference in its entirety.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

What is claimed is:

1. A conformable multi-arm optical catheter, comprising:
a proximal end;
a distal end;
a central axis;
a proximal catheter segment;
a distal interrogation section extending from the distal end of the proximal catheter segment, wherein the interrogation section comprises at least two flexible probe arms that in an unconstrained state radially bow out from the central axis and then, proceeding distally, bow back toward the central axis of the catheter; and
a distal insertion segment connected to the distal ends of the probe arms and providing a guidewire lumen so that the distal insertion segment is slideably engageable with the guidewire,
wherein each probe arm comprises at least one optical fiber entering the probe arm and terminating at or near the most radially extendable portion of the probe arm in a side-viewing configuration or assembly to form a side-viewing optical probe element capable of transmitting and collecting light.

2. The catheter of claim 1, wherein the distal ends of the probe arms are fixably connected to the distal insertion segment.

3. The catheter of claim 1, wherein the proximal catheter segment comprises a guidewire lumen for a guidewire.

4. The catheter of claim 1, wherein the distal insertion segment is configured to slideably surround the guidewire.

5. The catheter of claim 1, wherein the catheter is sized and configured for intravascular interrogation of a blood vessel wall.

6. The catheter of claim 5, wherein the blood vessel wall is a human coronary artery wall.

7. The catheter of claim 1, further comprising a preformed basket reinforcement element.

8. The catheter of claim 1, wherein at least the optical probe elements of the probe arms are enclosed in a polymeric material having an least substantially non-discernable Raman scattering signal in one or more preselected wavenumber regions used for analysis of a target and being adequately transparent to excitation light delivered via the optical probe element to Raman-scattered light collected from the illuminated target in the preselected wavenumber range by the optical fiber assembly

9. The catheter of claim 8, wherein the preselected wave number range is or is within the high wavenumber region.

10. The catheter of claim 2, wherein at least the optical probe elements of the probe arms are enclosed in a polymeric material having an least substantially non-discernable Raman scattering signal in one or more preselected wavenumber regions used for analysis of a target and being adequately transparent to excitation light delivered via the optical probe element to Raman-scattered light collected from the illuminated target in the preselected wavenumber range by the optical fiber assembly

11. The catheter of claim 10, wherein the preselected wave number range is or is within the high wavenumber region.

12. The catheter of claim 1, wherein the at least two flexible probe arms consist of six radially spaced, flexible probe arms.

13. An optical intravascular catheter system, comprising:
an optical catheter according to claim 1;
a light source in optical communication with the optical probe elements of the probe arm and suitable for generating Raman spectra; and
a Raman spectrometer in optical communication with the optical probe elements of the probe arm.

14. The system of claim 13, wherein the distal ends of the probe arms are fixably connected to the distal insertion segment.

15. The system of claim 13, further comprising at least one computer processor.

16. The system of claim 13, wherein at least the optical probe elements of the probe arms are enclosed in a polymeric material having an least substantially non-discernable Raman scattering signal in one or more preselected wavenumber regions used for analysis of a target and being adequately transparent to excitation light delivered via the optical probe element to Raman-scattered light collected from the illuminated target in the preselected wavenumber range by the optical fiber assembly.

17. The system of claim 16, wherein the preselected wave number range is or is within the high wavenumber region.

18. The system of claim 13, wherein at least the optical probe elements of the probe arms are enclosed in a polymeric material having an least substantially non-discernable Raman scattering signal in one or more preselected wavenumber regions used for analysis of a target and being adequately transparent to excitation light delivered via the optical probe element to Raman-scattered light collected from the illuminated target in the preselected wavenumber range by the optical fiber assembly.

19. The system of claim 18, wherein the preselected wave number range is or is within the high wavenumber region.

20. The system of claim 13, wherein the at least two flexible probe arms consist of six radially spaced, flexible probe arms.
